(19) United States
(12) Patent Application Publication
(10) Pub. No.: US 2025/0134536 A1
(43) Pub. Date: May 1, 2025

(54) PATIENT-CUSTOMIZED GUIDE, ELECTRONIC APPARATUS FOR PROVIDING THE SAME AND METHOD THEREOF

(71) Applicant: SEEANN Solution Co., Ltd., Incheon (KR)

(72) Inventors: Anna SEO, Incheon (KR); Hakjong NOH, Incheon (KR); Hyunho OK, Incheon (KR); Heedong LEE, Gyeonggi-do (KR)

(73) Assignee: SEEANN Solution Co., Ltd., Incheon (KR)

(21) Appl. No.: 18/946,423

(22) Filed: Nov. 13, 2024

Related U.S. Application Data
(63) Continuation of application No. PCT/KR2024/009758, filed on Jul. 9, 2024.

(30) Foreign Application Priority Data
Oct. 27, 2023 (KR) 10-2023-0145511
Oct. 27, 2023 (KR) 10-2023-0145512

(51) Int. Cl. A61B 17/16

(52) U.S. Cl. CPC A61B 17/1684

(57) ABSTRACT

Provided is a combined guidance for reaming and cutting, including: a reamer guidance configured to guide reaming of a humerus; a first humeral guidance press-fitted with the reamer guidance and having a surface tailored to a humerus head; a second humeral guidance having a surface tailored to an intertubercular groove of the humerus head; and a cutting guidance mutually coupled to the reamer guidance, the first humeral guidance, and the second humeral guidance.

### CROSS-REFERENCE TO RELATED APPLICATION

The present application is a continuation of International Patent Application No. PCT/KR2024/009758, filed on Jul. 9, 2024, which is based upon and claims the benefit of priority to Korean Patent Application No. 10-2023-0145511, filed on Oct. 27, 2023 and Korean Patent Application No. 10-2023-0145512, filed on Oct. 27, 2023. The disclosures of the above-listed applications are hereby incorporated by reference herein in their entirety.

### TECHNICAL FIELD

The present disclosure relates to a patient-customized guide, an electronic apparatus for providing the same, and a method thereof, and more specifically, to an electronic apparatus for providing a collaborative service between an operator and a user in three-dimensional modeling for designing a patient-customized guide to ensure safer surgeries, and a method thereof.

### BACKGROUND ART

As medical technology advances, medical services are progressively diversifying and more specialized. Additionally, with the development of electronic technology, there is an increasing trend to provide medical services using electronic technology. The use of electronic technology may include various acts of utilization, such as not only applying electronic technology to devices for direct medical treatment, but also providing remote medical services or selling various items on an e-commerce web page. These medical services are intended for use in the design and manufacturing of patient-customized medical devices. However, since medical services deal with human lives and health, it is required to carefully consider various factors related to each service, such as physical characteristics of individual patients, and determine details of the service provided.

An artificial joint is a medical device designed to restore the original function of a joint, in which the joint cartilage is damaged, deformed, or fractured due to degenerative arthritis, age-related disease, autoimmune disease, trauma, or the like, by removing the joint and inserting an artificial prosthesis (an implant) into a joint plane and surroundings of the joint plane.

With an artificial joint replacement surgery, which is a process of inserting an artificial joint into the human body, it is possible to restore the normal function of the joint in the area of discomfort. Typically, a shoulder artificial joint replacement surgery may be performed when the shoulder joint is damaged, conservative treatments are no longer effective, and there is severe pain.

However, since such surgeries have high risk and even minor errors may cause irreversible damage to the patient, it is important to accurately replace an artificial joint specific to each individual patient. This leads to an introduction of a surgery guidance that is designed based on each individual's different anatomy and increases the accuracy of artificial joint implantation. In other words, a patient-customized surgical guidance for an artificial joint replacement surgical implant needs to be designed in advance in a three-dimensional space. Therefore, today, a technology of establishing surgical plans based on patient's medical images (computed tomography: CT) and manufacturing a surgical guidance using 3D printing is advancing day by day.

In addition, since implantation methods vary depending on the surgeon performing the surgery, the present disclosure aims to provide a customized medical device that allows each surgeon's opinions to be included in the design of the guidance. In this regard, reference may be made to the related art documents KR20200117118A or KR20110087220A.

### DISCLOSURE

### Technical Problem

The present invention is directed to providing an electronic apparatus for providing a collaborative service between an operator and a user in relation to three dimensional (3D) modeling for designing or manufacturing a patient-customized body model or a patient-customized medical device in relation to provision of a medical service.

The present invention is directed to designing a guidance in a virtual three-dimensional space, the guidance being designed to fix an implant such that the implant is inserted in the most secure and accurate position in a virtual three-dimensional space.

In addition, the present invention is directed to providing a guidance capable of providing the position and the like that allow a guidance to be inserted into the most correct site, and also providing a guidance that incorporates the feedback of the operating surgeon

The technical challenges that this embodiment aims to achieve are not limited to the technical challenges described above, and other technical challenges may be inferred from the following embodiments.

### Technical Solution

According to an aspect of the present invention, there is provided a combined guidance for reaming and cutting, including: a reamer guidance configured to guide reaming of a humerus; a first humeral guidance press-fitted with the reamer guidance and having a surface tailored to a humerus head; a second humeral guidance having a surface tailored to an intertubercular groove of the humerus head; and a cutting guidance mutually coupled to the reamer guidance, the first humeral guidance, and the second humeral guidance.

The reamer guidance may include: a first screw hole for fastening with the first humeral guidance; a reamer insertion hole for reaming the humerus head; and a second screw hole for fastening with the cutting guidance, wherein the first humeral guidance may include a fastening groove for press-fitting with the reamer guidance.

The cutting guidance may include: a plurality of pin holes for fixation to the humerus; a sawing hole for sawing the humerus head as planned; and a third screw hole for fastening with the second humeral guidance, wherein the second humeral guidance may include: a groove for fastening with the third screw hole of the cutting guidance;
and a plurality of pin holes for fixation to the humerus.

The second screw hole of the reamer guidance for fastening with the cutting guidance may have an elongated shape that allows adjustment according to a skeletal size of the humerus.

The reamer guidance and the cutting guidance may include metal, and the first humeral guidance and the second humeral guidance may include plastic.

According to an aspect of the present invention,
there is provided a method of designing a guidance specific to a patient, the method including: designing a reamer guidance configured to guide reaming of a humerus; designing a first humeral guidance interference-fitted with the reamer guidance and having a surface tailored to a humerus head; designing a second humeral guidance having a surface tailored to an intertubercular groove of the humerus head; and designing a cutting guidance mutually coupled to the reamer guidance, the first humeral guidance, and the second humeral guidance.

The method may include, before the designing of the reamer guidance, the first humeral guidance, the second humeral guidance, and the cutting guidance; receiving image information about a body part of a target patient; performing 3D modeling based on the received image information; and providing a 3D modeling manufacturing function of a patient-customized medical device based on information about the 3D modeling.

The designing of the reamer guidance configured to guide reaming of the humerus and the designing of the cutting guidance mutually coupled to the reamer guidance, the first humeral guidance, and the second humeral guidance may include displaying, to an operator, predetermined specification information of the reamer guidance and predetermined specification information of the cutting guidance for selection.

The method may further include, when the operator inputs the specification information of the reamer guidance and the cutting guidance, and feedback information of the designed first humeral guidance and the designed second humeral guidance, modifying the design of the guidance based on the specification information of the reamer guidance and the cutting guidance, and the feedback information of the first humeral guidance and the second humeral guidance that are input by the operator.

According to an aspect of the present invention, there is provided an electronic apparatus for executing the method on a computer, the electronic apparatus including a transceiver, a memory storing instructions, and a processor.

According to an aspect of the present invention, there is provided a combined guidance for guiding positioning and implant installation, including: a positioning pin guidance configured to guide a position of an implant on a cut surface of a humerus that is subjected to cutting; and a rail guidance seated on the positioning pin guidance, and having a hole provided for fixing and coupling the rail guidance to a periphery of a head of the humerus that is subjected to cutting.

The positioning pin guidance may include: a handle allowing a user to insert or remove the positioning pin guidance into or from a reamed hole of the humerus; a body allowing the positioning pin guidance to be seated on the cut surface of the humerus; and a pin configured to be inserted into a pre-reamed hole to secure a position.

The rail guidance may include: an internal implant rail portion configured to engage with an implant or a broach desired to be installed; an external body configured to support the internal implant rail portion; and a plurality of holes provided for fixing and coupling the rail guidance to the periphery of the head of the humerus that is subjected to cutting.

The positioning pin guidance may be removable when the rail guidance is fixed and coupled to the periphery of the head of the humerus that is subjected to cutting.

The plurality of holes of the rail guidance may be pre-designed with pin insertion angles of the holes and hole depths of the holes.

According to an aspect of the present invention, there is provided a method of designing a guidance specific to a patient, the method including: designing a positioning pin guidance configured to guide a position of an implant on a cut surface of a humerus that is subjected to cutting; and designing a rail guidance configured to be seated on the positioning pin guidance, and having a hole provided for fixing and coupling the rail guidance to a periphery of a head of the humerus that is subjected to cutting.

The method may further include, before the designing of the positioning pin guidance and the rail guidance, receiving image information about a body part of a target patient; performing 3D modeling based on the received image information; and providing a 3D modeling manufacturing function of a patient-customized medical device based on information about the 3D modeling.

The designing of the positioning pin guidance configured to guide the position of the implant on the cut surface of the humerus, and the designing of the rail guidance configured to be seated on the positioning pin guidance and having the hole provided for fixing and coupling the rail guidance to the periphery of the head of the humerus that is subjected to cutting may include displaying, to an operator, predetermined specification information of the positioning pin guidance and predetermined specification information of the rail guidance for selection.

The designing of the rail guidance configured to be seated on the positioning pin guidance and having the hole provided for fixing and coupling the rail guidance to the periphery of the head of the humerus that is subjected to cutting may include inputting: by an operator, positions of a plurality of holes provided for fixing and coupling the rail guidance to the periphery of the head of the cut humerus, pin insertion angles of the holes, and hole depths of the holes; and modifying the design of the guidance based on information about the positions of the plurality of the holes, the pin insertion angles of the holes, and the hole depths of the holes that are input by the operator.

According to an aspect of the present invention, there is provided an electronic apparatus for executing the above described method of designing a guidance specific to a patient on a computer, the electronic apparatus including a transceiver, a memory storing instructions, and a processor.

Specific details of other embodiments are included in the detailed description and drawings.

### Advantageous Effects

As is apparent from the above, the present disclosure is implemented to provide a collaborative service between an operator and a user through a three dimensional (3D) modeling task for designing or manufacturing a patient-customized body model or medical device in relation to provision of a medical service, thereby providing a patient-customized body model or medical device in which user requirements are accurately reflected, preventing incorrect or inadequate medical services from being provided.

In addition, the collaborative service according to the present disclosure can enable exchange of detailed feedback regarding the results of 3D modeling while enhancing the convenience of feedback exchange.

The method disclosed herein is implemented to provide a customized humeral guidance for the implant to be properly implanted in a patient-customized position in an artificial joint replacement surgery, thereby reducing the risk in actual replacement surgeries.

In addition, the method disclosed herein is implemented to design a guidance that is intuitively identified with respect to the patient's humerus, thereby controlling errors occurring due to environmental differences between a virtual 3D space and an actual space.

The effects of the present disclosure are not limited to those described above, and other effects not described above is clearly understood by those skilled in the art from the above detailed description.

### Description of Drawings

FIG. 1 is a schematic block diagram illustrating a system for providing a collaborative service between an operator and a user in 3D modeling and design for designing or manufacturing a patient-customized body model or a patient-customized medical device according to an embodiment;

FIG. 2 is a flowchart for describing an operation of an electronic apparatus in a system for providing a collaborative service according to an embodiment;

FIG. 3 is a flowchart for a process in which an electronic apparatus prepares (generates) a patient-customized humeral guidance in a system for providing a collaborative service according to an embodiment;

FIG. 4 is a configuration block diagram of an electronic apparatus according to an embodiment;

FIG. 5 is a perspective view of a reamer guidance according to an embodiment;

FIG. 6 is a perspective view of a cutting guidance according to an embodiment;

FIG. 7 is a side perspective view illustrating a state in which a first humeral guidance is installed on the humerus according to an embodiment;

FIG. 8 is a side perspective view illustrating a state in which ha second humeral guidance is installed on the humerus according to an embodiment;

FIG. 9 is a perspective view illustrating a positioning pin guidance according to an embodiment;

FIG. 10 is a perspective view illustrating a state in which a rail guidance is installed on the humerus according to an embodiment;

FIG. 11 is a perspective view illustrating a state in which a first humeral guidance is coupled to a reamer guidance according to an embodiment;

FIG. 12 is a perspective view illustrating a state in which a second humeral guidance is coupled to a cutting guidance according to an embodiment;

FIG. 13 is a perspective view illustrating a state in which the first humeral guidance and the reamer guidance coupled as shown in FIG. 11 are installed on the humerus according to an embodiment;

FIG. 14 is a perspective view illustrating a state in which the second humeral guidance and the cutting guidance coupled as shown in FIG. 12 are installed on and coupled to the humerus on which the first humeral guidance and the reamer guidance are installed as shown in FIG. 13;

FIG. 15 is a perspective view illustrating an operation of inserting a reamer after the first humeral guidance, the reamer guidance, the second humeral guidance, and the cutting guidance are installed and coupled on the humerus as shown in FIG. 14;

FIG. 16 is a perspective view illustrating a cutting operation of a saw after the first humeral guidance, the reamer guidance, the second humeral guidance, and the cutting guidance are installed and coupled on the humerus as shown in FIG. 14;

FIG. 17 is a perspective view illustrating an operation in which a positioning pin according to an embodiment as shown in FIG. 9 is inserted into the humerus, and a rail guidance is seated on the positioning pin;

FIG. 18 is a perspective view illustrating an operation in which a broach is inserted along the rail guidance according to an embodiment; and

FIG. 19 is a perspective view illustrating an operation in which a real implant is inserted along the rail guidance according to an embodiment.

### MODES OF THE INVENTION

Although terms used herein are selected from among general terms that are currently and widely used in consideration of functions in the exemplary embodiments, these may be changed according to intentions or customs of those skilled in the art or the advent of new technology. However, when a specified term is defined and used in an arbitrary sense, a meaning of the term will be described in the specification in detail. Accordingly, the terms used herein are not to be defined as simple names of the components but should be defined based on the actual meaning of the terms and the whole context throughout the present specification.

Throughout the specification, the term “comprises” or “includes” and/or “comprising” or “including” means that one or more other components may not be further excluded unless context dictates otherwise. In the specification, the term “part” or “module” refers to a unit for processing at least one function or operation that may be implemented in hardware, software, or a combination thereof.

As used herein, expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, or all of a, b, and c.

Meanwhile, a “terminal” described below may be implemented as a computer or a portable terminal capable of connecting to a server or another terminal through a network. Here, the computer may include, for example, a notebook computer, a desktop computer, a laptop PC, and the like, each of which is equipped with a WEB Browser. The portable terminal is a wireless communication device, and may include: all types of handheld devices based on wireless communication, such as a communication-based terminal, e.g., an international mobile telecommunication (IMT), a code division multiple access (CDMA), a w-code division multiple access (W-CDMA), a long term evolution (LTE), and the like, a smart phone, and a table PC.

Although embodiments of the present disclosure will be described in detail with reference to the accompanying drawings in order to enable those skilled in the art to easily practice the disclosure, the present disclosure may be embodied in many different forms and should not be construed as being limited to the embodiments set forth herein.

Hereinafter, embodiments of the present invention will be described in detail with reference to the attached drawings.

In the description of the embodiments, the detailed description of related known functions or constructions will be omitted herein to avoid making the subject matter of the present disclosure unclear.

In the accompanying drawings, sizes of elements may be exaggerated, reduced or schematically illustrated for the sake of convenience in description, and may not reflect actual shapes of each element. In addition, the same drawing reference numerals will be understood to refer to the same elements, features, and structures.

The advantages and features of the present invention and ways of achieving them will become readily apparent with reference to descriptions of the following detailed embodiments in conjunction with the accompanying drawings. However, the present invention is not limited to such embodiments and may be embodied in various forms. The embodiments to be described below are provided only to complete the disclosure of the present invention and assist those of ordinary skill in the art in fully understanding the scope of the present invention, and the scope of the present invention is defined only by the appended claims. In connection with assigning reference numerals to elements in the drawings, the same reference numerals are used for designating the same elements throughout the specification

In this case, the combinations of blocks and flowchart illustrations in the process flow charts may be performed by computer program instructions. These computer program instructions may be loaded into a processor of a general purpose computer, special purpose computer, or other programmable data processing equipment so that the instructions, which may be executed by a processor of a computer or other programmable data processing equipment may generate means for performing the functions described in block(s) of the flowchart. These computer program instructions may also be stored in a computer usable or computer readable memory capable of directing a computer or other programmable data processing apparatus to implement the functionality in a particular manner so that instructions stored in the computer usable or computer readable memory may also produce a manufacturing item containing instruction means for performing the functions described in block(s) of the flowcharts. Computer program instructions may also be loaded into a computer or other programmable data processing equipment so that a series of operating steps are performed on the computer or other programmable data processing equipment to generate a computer-executable process and execute the computer or other programmable data processing equipment, providing steps for executing the functions described in block(s) of the flow charts.

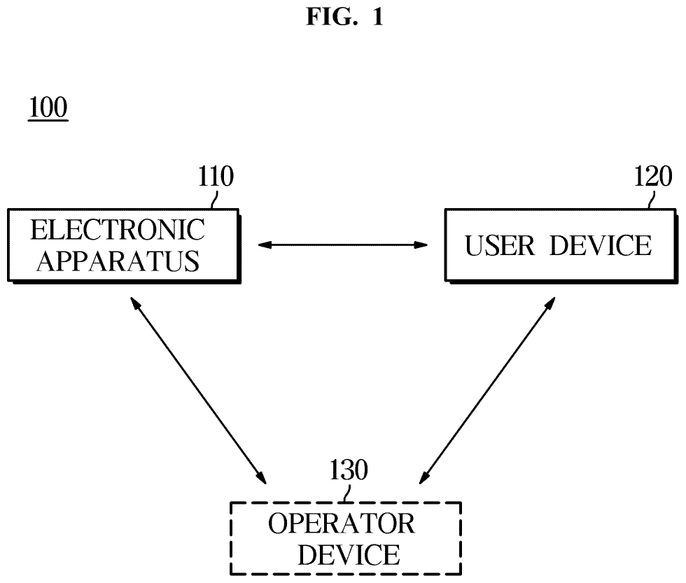

FIG. 1 is a schematic block diagram illustrating a system for providing a collaborative service between an operator and a user in 3D modeling for designing or manufacturing a patient-customized body model or a patient-customized medical device in relation to a medical service according to an embodiment.

According to various embodiments, a system 100 for providing a collaborative service includes an electronic apparatus 110 and a user device 120, and may further include an operator device 130 according to an embodiment. According to an embodiment, the system 100 for providing a collaborative service may further include a network for supporting information transmission and reception between at least a part of the electronic apparatus 110, the user device 120, and the operator device 130.

According to an embodiment, the medical service may include a service for manufacturing a body organ model, a medical device, a body assistance device, a surgical guidance, etc., which are manufactured in a user-customized manner using a three-dimensional modeling technique. For example, the medical service may involve providing a three-dimensional simulation model that reflects the shape, condition, etc. of a user's body that is subject to various treatments, such as procedures, surgeries, and the like; or may involve reconstructing or treating a patient's body part that has been damaged or deformed due to various factors, such as birth conditions, diseases, tumors, personal injuries, or failure of previous surgeries; or may involve providing an assistive device to assist a body part that has been damaged or deformed; or may involve manufacturing a guidance required for surgery, but the present disclosure is not limited thereto.

Specifically, the patient-customized medical device according to an embodiment may be a surgical guidance for a graft or implant transplant surgery, such as a guidance for a shoulder joint replacement surgery, including a center-pin, a base plate, a screw, a reamer guidance, a cutting guidance, a rail guidance, and the like, but is not limited thereto.

Each of the electronic apparatus 110, the user device 120, and the operator device 130 may include a transceiver, a memory, and a processor. In addition, each of the electronic apparatus 110, the user device 120, and the operator device 130 refers to a unit that may process at least one function or operation, and may be implemented as hardware or software, or a combination of hardware and software. Meanwhile, throughout the embodiments, each of the electronic apparatus 110, the user device 120, and the operator device 130 is referred to as a separate device or server, but may be a structure divided based on a logical aspect, and at least a part of the electronic apparatus 110, the user device 120, and the operator device 130 may be implemented by a separate function in one device or server.

According to an embodiment, the electronic apparatus 110, the user device 120, and the operator device 130 may include a plurality of computer systems or computer software implemented on a network server. For example, at least a part of the electronic apparatus 110, the user device 120, and the operator device 130 may refer to a computer system and computer software that are connected to a lower level device that may communicate with other network servers through a computer network, such as an intranet or the Internet, receive a request for task execution, perform the task, and provide a result of the execution. In addition, at least a part of the electronic apparatus 110, the user device 120, and the operator device 130 may be understood in a broad concept that includes a series of applications that may operate on a network server and various databases built inside or on other connected nodes. For example, at least a part of the electronic apparatus 110, the user device 120, and the operator device 130 may be implemented using a network server program that is provided in various ways according to an operating system, such as DOS, Windows, Linux, UNIX, or MacOS.

The electronic apparatus 110 is a device that configures various types of information and provides the information. The electronic apparatus 110 may provide the configured information as a web page, an application screen, etc. The electronic apparatus 110 may be a device serving to provide users and operators with various types of information, such as information related to the provision of medical services. Therefore, the electronic apparatus 110 according to an embodiment may be configured as a server (not shown).

The electronic apparatus 110 acquires one or more pieces of suggestion information related to the provision of a patient-customized body model or medical device, and provides the acquired one or more pieces of suggestion information to the user. The electronic apparatus 110 may generate at least a part of the one or more pieces of suggestion information, and may also receive at least a part of the one or more pieces of suggestion information from the operator. In addition, the electronic apparatus 110 may acquire suggestion information according to various embodiments.

The electronic apparatus 110 receives response information regarding the one or more pieces of suggestion information from the user. The received response information may include various types of information, such as an approval for the suggestion information, a rejection for the suggestion information, and a request for modification of the suggestion information. In addition, when there is an established rule regarding an interpretation of cases in which there is no separate response to a suggestion (for example, in the absence of a separate response, content of suggestion information is implicitly considered an approval), no separate response may also be considered an implicit response, and may be understood and interpreted as falling within the scope of the present disclosure.

When the response information includes a request for modification related to at least a part of the one or more pieces of the suggestion information, the electronic apparatus 110 initiates a process for modifying the at least a part of the one or more pieces of the suggestion information. In this regard, the electronic apparatus 110 may request the operator to modify the at least a part of the one or more pieces of the suggestion information, may provide a function for transmitting and receiving information between the user and the operator, and additionally, various embodiments may be present in relation to initiating a process for modifying the suggestion information.

The user may be an entity that has requested the designing or manufacturing of a patient-customized body model or medical device, and may include various entities related to the provision of medical services using the patient-customized body model or medical device. As an example, the user may include a person who provides medical services, such as a medical professional (a doctor, particularly a “an operating surgeon”). As another example, the user may include a manager who manages and supervises whether there are any problems with the provision of medical services, but it is not limited to managers. In addition, the user according to the present disclosure is not limited to those who provide medical services, and it should be understood that the disclosure may include an embodiment in which a user makes a confirmation, and then the provision of medical services is finally performed by the electronic apparatus 110 or by the operator.

The user device 120 is a device that is manipulated and managed by the user. The user device 120 may receive a user input from the user, etc., or receive information from the electronic apparatus 110, etc., and perform an operation corresponding to the user input or the information. For example, the user device 120 may receive one or more pieces of suggestion information from the electronic apparatus 110, and provide the one or more pieces of suggestion information to the user. The user device 120 may provide the information received from the electronic apparatus 110 as it is, or may additionally process the information (for example, converting the information received from the electronic apparatus 110 such that the information may be output to an output device, such as a screen or speaker, and outputting the converted information) and provide the processed information. According to an embodiment, the user device 120 may further perform various operations, such as receiving a response from the user, such as a request for modification, and providing the response to the electronic apparatus 110.

The operator may include an entity that transmits at least a part of the one or more pieces of suggestion information to the electronic apparatus 110, or transmits information that serves as the basis for generating at least a part of the one or more pieces of suggestion information to the electronic apparatus 110 (in which case, the suggestion information may be finally generated in the electronic apparatus 110). As an example, the operator may include a 3D modeler that three-dimensionally models a medical device or a body model, which is to be applied to or inserted into a patient, and transmits the result.

The operator may include, but is not limited to, a natural person, and may include a comprehensive entity, such as a business related to providing medical services. However, for convenience of description, an embodiment in which the operator is a natural person is described below, but the following descriptions may be applied to various types of operators.

The operator device 130 is a device manipulated and managed by the operator. The operator device 130 may receive a user input from the operator, etc., or receive information from the electronic apparatus 110, etc., and perform operations corresponding to the user input or the information. For example, the operator device 130 may receive basic information, such as information related to a target patient, from the electronic apparatus 110 and the user device 120, and provide the received basic information to the operator. The operator device 130 may provide the received information as it is, or may additionally process the received information (for example, converting the received information such that the information may be output on an output device, such as a screen or speaker, and outputting the converted information) and provide the processed information. In addition, the operator device 130 may receive an input related to the one or more pieces of suggestion information from the operator and provide the received input to the electronic apparatus 110, and may further perform various operations, such as transmitting and receiving various types of information related to a modification process.

The user device 120 and the operator device 130 may include a computer device, a mobile communication terminal, a server, etc. The user device 120 and the operator device 130 may include or be connected to an input device, such as a touch pad, a mouse, a keyboard, etc. for receiving a user input. In addition, the user device 120 and the operator device 130 may include or be connected to an output device, such as a screen, a speaker, an interface device, etc. for providing information to the user. Furthermore, the input device and the output device of the user device 120 or the operator device 130 may be configured as one part or may be interconnected with each other. For example, an interface for receiving a user input may be displayed on the user device 120 or the operator device 130. The user device 120 and the operator device 130 may be provided using various terminals, for example, a computer, a tablet PC, or a mobile phone.

A method of performing a series of operations for providing a collaborative service between a user and an operator according to various embodiments may be implemented by a single physical device, or may be implemented by a plurality of physical devices that are organically combined to each other. For example, some components included in the system 100 for providing a collaborative service may be implemented by one physical device, and some other components may be implemented by another physical device. For example, the one physical device may be implemented as a part of the electronic apparatus 110, and the other physical device may be implemented as a part of the user device 120 or a part of another external device. In some cases, components included in the system 100 for providing a collaborative service may be distributed and placed in different physical devices, and the distributed components may be organically combined to perform the functions and operations of the system 100 for providing a medical service. For example, the electronic apparatus 110 in the present specification may include at least one sub-device, and some operations described as being performed by the electronic apparatus 110 may be performed by a first sub-device, and some other operations may be performed by a second sub-device.

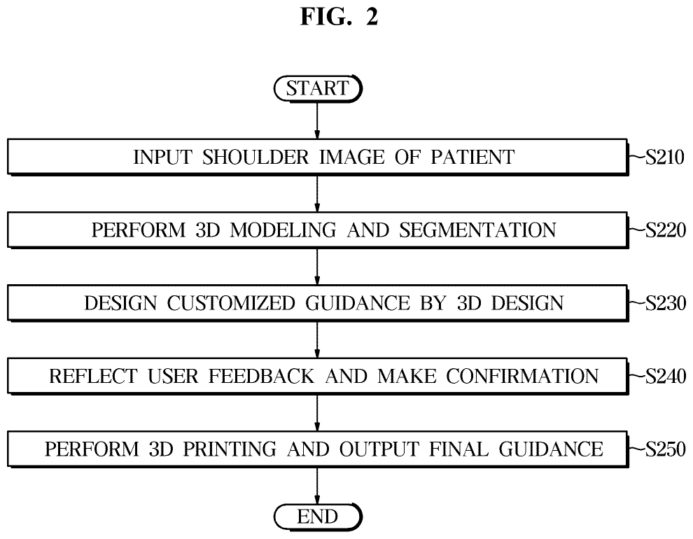

FIG. 2 is a flowchart for describing an operation of an electronic apparatus 110 in a system for providing a collaborative service according to an embodiment.

First, the electronic apparatus 110 receives medical data including shoulder image information of a patient (S210). In this case, the electronic apparatus 110 receives, from the user device 120 or the operator device 130, a shoulder image, which may include a shoulder magnetic resonance imager (MRI) image or a shoulder computer tomography (CT) image, but is not limited thereto.

That is, the medical data may include at least one of image data, audio data, and time-series data. The image data includes all image data that is obtained by capturing or measuring a patient's affected area through an examination device, converting the result into an electrical signal and outputting the electrical signal. The image data may include image data that constitutes each frame of a video that is continuously recorded over time by a medical imaging device. For example, the image data may include ultrasound examination image data, image data by an MRI device, CT tomography image data, X-ray image data, and the like. In addition, when audio data is converted into electrical signals and the electrical signals are output as an image in the form of a graph, or when time-series data is represented as visualized materials, such as a graph, the image or materials may be included in the above-described image data. For example, the medical data may include CT images. The above-described examples of the medical data are merely illustrative and do not limit the present disclosure.

Next, the electronic apparatus 110 may, when preprocessing the acquired medical data, segment a target area from the acquired medical data (S220). For example, the electronic apparatus 110 may segment only the humerus bone to manufacture a humeral guidance. That is, the electronic apparatus 110 generates a 3D model of a part required for designing and manufacturing a customized humeral guidance from the acquired medical image data and corrects the generated 3D model into a designable form.

Next, the electronic apparatus 110 designs a customized humeral guidance using the 3D model acquired from the medical image data (S230). Specifically, the electronic apparatus 110 designs a first humeral guidance that fits a humerus head of a humerus extracted from the personal medical image, and a second humeral guidance that fits an intertubercular groove portion of the humerus. In addition, the electronic apparatus 110 may recommend specification information of a reamer guidance, a cutting guidance, a positioning pin guidance, and a rail guidance based on skeletal information of the patient acquired from the medical image data.

Depending on the order, the first humeral guidance and the second humeral guidance may be designed to match the specification information of the reamer guidance, the cutting guidance, the positioning pin guidance, and the rail guidance that are selected.

Here, the selected reamer guidance, cutting guidance, positioning pin guidance, and rail guidance may be in a pre-manufactured form, and may be formed of metal. However, the present disclosure is not limited thereto, the selected reamer guidance, cutting guidance, positioning pin guidance, and rail guidance may also be generated each time by the electronic apparatus 110, similar to the first humeral guidance and the second humeral guidance.

As to be described below, the reamer guidance, the cutting guidance, the first humeral guidance, and the second humeral guidance form a first coupling guidance, and the positioning pin guidance and the rail guidance form a second coupling guidance, separately from the first coupling guidance, and are used in surgery. However, since the positioning pin guidance and the rail guidance are provided to be fastened to a cutting line/surface of the humerus generated by the first coupling guidance, handling the manufacturing methods, specification information, and the like thereof together in the electronic apparatus 110 according to the present disclosure may be beneficial in improving the convenience and stability of surgery through the use of the guidance.

Next, when the electronic apparatus 110 displays the 3D-designed customized guidance and the specification information of the plurality of pieces of guidance on the user device 120, the user who has checked the information inputs feedback (S240). For example, the user may check a 3D image in which the 3D-designed customized guidance and the plurality of pieces of guidance are fitted to the humerus, and input feedback to modify the position, direction, and angle of the individual guidance, or to modify the specifications of the guidance.

According to an embodiment, the response information from the user device 120 for the feedback may include image information obtained by manipulating 3D modeling of a patient-customized body part or 3D modeling of a patient-customized medical device. As an example, the response information may include 3D modeling information obtained by shift in the XYZ axis direction, zoom-in, zoom-out, rotation, or twist for all or a part of the 3D modeling. As another example, the response information may include 3D modeling information obtained by grouping all or a part of elements constituting the 3D modeling, and if needed, assigning the group a group name, or image information obtained by manipulating the 3D modeling information. When a plurality of elements are grouped into one group, the entire group may be shifted, zoomed-in/zoom-out, rotated. The 3D modeling image changed as described above may be projected to provide the user with continuous visual feedback.

According to an embodiment, when the response information includes a request for modifying at least a part of the one or more pieces of suggestion information, the electronic apparatus 110 initiates a process for modifying the at least a part of the one or more pieces of suggestion information. A request for modifying each of the one or more pieces of suggestion information may be a request for modifying the entire information, or may be a request for modifying only a part of the information. For example, in relation to providing a guidance for a joint replacement surgery, a user may provide information requesting a modification for a group of screws included in the suggestion information, or may approve or request a modification for individual screws.

In this regard, the input from the user device 120 corresponding to the modification request may include not only simply marking or clicking the position for which the modification is desired, but also cases of a text memo explicitly entering a phrase, such as “request to add 5 mm screw”, or an image memo in which a specific image is extracted and annotated with a phrase, such as “add 5 mm”. Alternatively, a modification request including specific modification details may also be made based on various methods, such as a voice chat and a text chat.

According to an embodiment, the response information may include information about all or a part of a medical device additionally designed by the user in response to the suggestion information about the 3D modeling of the patient-customized body part, and more specifically, the response information may include a 3D modeling image of all or a part of a medical device desired to be added by the user, information about a shape, a material, a color, a size, a weight, a price and the like of the medial device, and an additional manufacturing request that allows an operator to manufacture the medical device.

In greater detail with regard to initiating a process for modifying at least a part of the one or more pieces of suggestion information, the electronic apparatus 110 according to an embodiment may request the operator to modify at least a part of the one or more pieces of suggestion information. In this case, the operator to whom the electronic apparatus 110 requests modification of the at least a part of the one or more pieces of suggestion information may be the same as or different from the operator who has transmitted the one or more pieces of suggestion information, and whether the operator is the same person or not does not limit the scope of disclosure. For example, in order to increase the task efficiency, the electronic apparatus 110 may request a modification from the operator who has transmitted the one or more pieces of suggestion information, but when the operator is absent or it is determined inappropriate to request a modification from the operator (e.g., the operator in question has a strong tendency to repeat similar mistakes), the modification may be requested from another operator.

The following description is made in relation that, for convenience of description, the operator requested to modify at least a part of one or more pieces of suggestion information is the same person as the operator who has transmitted the one or more pieces of suggestion information. However, this is only for convenience of description. The description may be applied even when the operator requested for modification of at least a part of the one or more pieces of suggestion information is different from the operator who has transmitted the one or more pieces of suggested information.

According to an embodiment, in relation to initiating a process for modifying at least a part of one or more pieces of suggestion information, the electronic apparatus 110 may provide a function for transmitting and receiving information between a user and an operator. For example, the function for transmitting and receiving information may include one or more functions among a function of providing a first interface to a user and a function of providing a second interface to an operator, and the electronic apparatus 110 may reflect at least a part of an input of the operator in the first interface in real time, and reflect at least a part of an input of the user in the second interface in real time such that real-time information transmission and reception may be achieved.

As a more specific example, the electronic apparatus 110 may provide the user with details of a real-time modification of the operator or a real-time task screen of the operator, and provide the user device 120 with a function that allows the user to input a voice chat, a text chat, a text memo, an image memo and the like in relation to the real-time modification of the operator such that the user may inform whether the modification is appropriate or may provide specific instructions regarding the modification

Furthermore, there are other various embodiments that provide a function of transmitting and receiving information between the user and the operator, such as the electronic apparatus 110 allows the user and the operator to perform a task together on a single shared screen, enabling the user to directly delete and change the content of the operator's task.

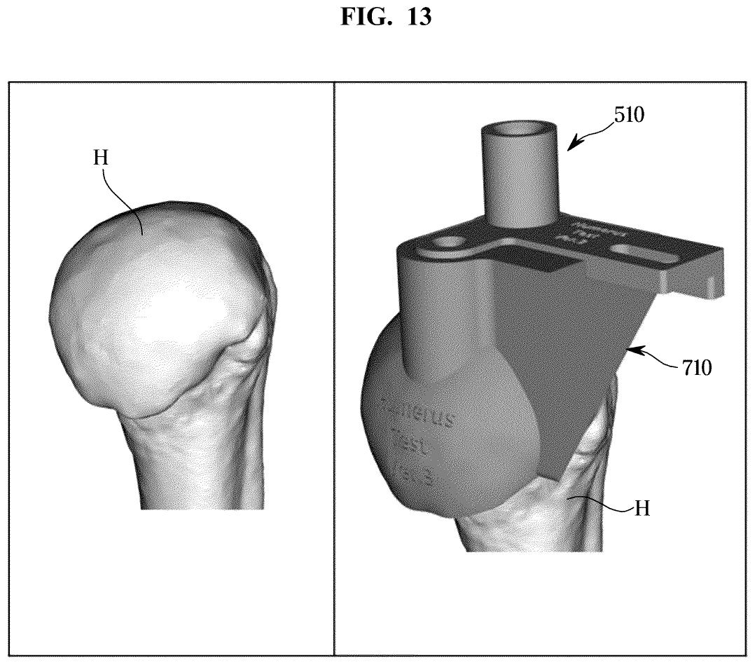

Regarding a process for modifying at least a part of the one or more pieces of suggestion information, the electronic apparatus 110 may provide a user's modification request to the operator device 130 in the same manner as shown in FIGS. 13 to 16. The operator may proceed with a modification based on the received modification request and provide modified suggestion information back.

According to an embodiment, the electronic apparatus 110 may group one or more pieces of suggestion information and response information corresponding thereto and store the one or more pieces of suggestion information and the response information corresponding thereto in a database. Here, the database may be understood as a broad concept that includes not only external devices distinct from the electronic apparatus 110, but also internal memories of the electronic apparatus 110, or a separate cloud service and the like.

The electronic apparatus 110 may acquire feedback information related to an operation of acquiring one or more pieces of suggestion information based on the information stored in the database. For example, in connection with a specific surgery, when one or more users repeatedly request that an implant or graft material should be inserted at a position slightly higher than the suggested position, the electronic apparatus 110 may acquire feedback information for suggesting a slightly higher position as an insertion position in the future. With such a process, the process associated with acquiring one or more pieces of suggestion information may be gradually improved.

Meanwhile, according to embodiments, the acquisition of feedback information may be achieved based on a machine learning algorithm. For example, as one or more parameters included in a neural network model for generating suggestion information are learned based on information stored in the database, the accuracy of generating suggestion information may be gradually improved.

According to an embodiment, the electronic apparatus 110 may acquire information about a result of a medical services provided from a user and the like. The information about the result of providing the medical service may include evaluation information related to the provided medical service. Alternatively, the electronic apparatus 110 may evaluate the provided medical service based on the information about the result of providing the medical service. In this regard, the evaluation of a medical service may be performed based on indicators, such as the objective quality of the medical service, the patient's subjective satisfaction with the medical service, and the efficiency of the medical service (in terms of cost, time and the like), but in the present disclosure, the scope of the evaluation is not limited thereto. In this regard, as an example, the electronic apparatus 110 may acquire a computed tomography (CT) image related to the provision of a medical service and evaluate the objective quality of the medical service based on the acquired CT image.

According to an embodiment, the electronic apparatus 110 may acquire feedback information related to an operation of acquiring the one or more pieces of suggestion information based on the result information. For example, in relation to a particular surgery, when an implant or graft material is inserted according to a suggested position, but the surgery is not completely successful, a suggestion process may be improved such that the suggested position is slightly adjusted in the future.

According to an embodiment, the acquisition of feedback information based on the result information may be achieved based on a machine learning algorithm.

According to an embodiment, the electronic apparatus 110 may, in addition to the one or more pieces of suggestion information and the response information corresponding thereto, further group the result information corresponding thereto and store the grouped information in the database, and may further group the feedback information and store the grouped information in the database, that is, the combination of pieces of information to be grouped and stored in the database may be determined in various ways.

Next, returning to FIG. 2, the electronic apparatus 110 reflects feedback information (suggestion information) from the user device 120, and upon a confirmation of the information being received from the user, 3D prints and outputs the final guidance (S250).

As described above, the electronic apparatus 110 may provide a 3D modeling image, which may include a 3D modeling image related to at least a body part of the patient based on medical image data of the patient's body, and more specifically, include a 3D modeling image that accurately represents a target body part or organ part of the patient based on a magnetic resonance imager (MRI) and computer tomography (CT) image, as described above. As an example, when the medical service is “an insertion/replacement surgery of an implant or graft material,” the electronic apparatus 110 may consider the implant or graft material to be inserted, or the structure, the presence/absence of deformation, and the degree of deformation of a patient's body part to which the guidance for the surgery is applied, and suggest, to the user, information related to 3D modeling of the corresponding body part based on the considered conditions.

According to an embodiment, the one or more pieces of suggestion information may include information for suggesting one or more items selected from a shape, a material, a color, a transparency, a size, a weight, a price, a manufacturing timeline, an application position, an angle, a depth, and a specific manufacturing operation of a patient-customized body model or a patient-customized medical device. Here, visual representation information of the body model or the medical device included in the suggestion information may include various forms, such as point clouds, wire frames, textured frames, or 3D modeling. In the case of a color and a transparency, a patient's body part may be represented with different colors or transparencies for each tissue, and a medical device may be represented with different colors or transparencies for each device that forms the medical device. In the case of a size, size/length information may be provided for the entirety or a selected portion of the corresponding body part or the medical device. Meanwhile, as an example of the suggestion information, the electronic apparatus 110 may include information for suggesting one or more items selected from a shape, a material, a color, a transparency, a size, a weight, a price, a manufacturing timeline, and a specific manufacturing operation of 3D modeling of a target body model of a patient. More specifically, the suggestion information may include a 3D modeling image of a shoulder joint and shoulder skeleton, and a segmented humerus of a patient, while including information about the shape of the shoulder joint and shoulder skeleton, and the segmented humerus, as well as information about a 3D image in which the transparency may be adjusted at various levels, or length, width, and thickness information of each region of the shoulder joint and shoulder skeleton, and the segmented humerus.

As another example, the electronic apparatus 110 may include information for suggesting one or more items selected from a shape, a material, a color, a transparency, a size, a weight, a price, a manufacturing timeline, and a specific manufacturing operation of 3D modeling of an implant or a graft material to be used in an insertion/replacement surgery of the implant or graft material. As another example, the electronic apparatus 100 may, with regard to a surgery guidance for an insertion/replacement surgery of an implant or graft material, more specifically, a joint replacement surgery implant or surgery guidance, (for example, a center-pin, a base plate and a screw, a customized guidance and a plurality of pieces of prototype guidance), suggest, to the user, specification (size) information, a material, a manufacturer of commercially available parts to be used for the manufacturing, a price, and a specific manufacturing operation thereof. For example, the suggestion information may include information distinguished by various colors for each component (guidance).

According to an embodiment, the one or more pieces of suggestion information may include specific guidance information for a medical service that uses a patient-customized medical device. For example, a reamer guidance, a cutting guidance, a positioning pin guidance, and a rail guidance described below may be pre-manufactured in the form of metal with a plurality of specifications (such as sizes 1, 2, 3, etc.), and the electronic apparatus 110 may designate (recommend) and display each of the plurality of specifications based on the patient's skeletal information, and may three-dimensionally model a customized guidance that matches the designated guidance.

According to an embodiment, the one or more pieces of suggestion information may include simulation information regarding changes that may occur in the patient's body after the use of the patient-customized medical device. As an example, the one or more pieces of suggestion information may include information suggesting changes that may occur in the patient's body after use of a surgery guidance of an artificial joint (e.g., a shoulder joint) or after an insertion of an implant/graft.

For example, the one or more pieces of suggestion information may display simulation results, such as the angle of a fixing pin when a guidance is installed.

In addition, according to an embodiment, the one or more pieces of suggestion information may include information for providing a function of manufacturing a surgical guidance to be inserted into a patient, or may include suggestion information about an implant or a surgical guidance that is semi-automatically manufactured through 3D modeling. As an example, the one or more pieces of suggestion information may include information related to a function of semi-automatically manufacturing a cylinder with a center-pin or a screw, which is a guidance for a shoulder joint replacement surgery (such as a function of adjusting the number, the thickness, the angle, the color, and the like).

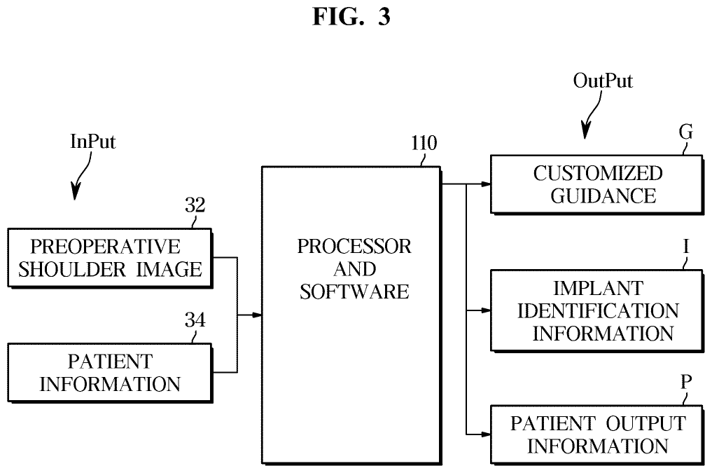

FIG. 3 is a flowchart for a process in which an electronic apparatus prepares (generates) a plurality of pieces of patient-customized humeral guidance in a system for providing a collaborative service according to an embodiment.

In FIG. 3, a method for the electronic apparatus 110 to form a customized guidance G is schematically illustrated. The customized (humeral) guidance G is formed as a patient-specific guidance by referring to preoperative images, such as MRI, CT images, etc. The electronic apparatus 110 receives an input INPUT and generates an output OUTPUT including the customized guidance G. The input INPUT includes a preoperative shoulder image 32 and patient (input) information 34. The preoperative shoulder image 32 may include an MRI image, a CT image, and other information that allow for rendering or characterizing of the humerus H in a position that is as close as possible to the head HH. For example, the preoperative shoulder image 32 may include the position and orientation of the anatomical neck and other features in the vicinity of the humerus H. The preoperative shoulder image 32 may include information about the position, shape, and orientation of the major tubercle, minor tubercle, surgical neck, bicipital groove BG, or other major landmarks of the humerus 12 of the humerus H. The patient input information 34 may include the patient's name, the shoulder being treated, and other information related to the past and future treatments and procedures.

The electronic apparatus 110 includes several modules capable of processing the input INPUT. The electronic apparatus 110 may include a function to select between a reverse implant configuration or an anatomical implant configuration, and a function to specify or select a stem size (STEM SIZE) of the humerus to be implanted into the humerus H. The electronic apparatus 110 may determine an insertion offset, which corresponds to the distance from the center of the resected surface to the center of the position of the implanted stem. These functions and other features may be determined and defined by the electronic apparatus 110 and may be included in the output of the electronic apparatus 110.

The output may include a customized humeral guidance G, implant identification information I, and patient output information P. The humeral guidance G that may be output from the electronic apparatus 110 may be a plan for forming the guidance or may be an actual guidance that is output by the electronic apparatus 110 through operation S250 of FIG. 2. In this case, examples of 3D printing include direct metal laser sintering (DMLS), fused deposition modeling (FDM), fused filament fabrication (FFF), and electron beam melting (EBM). In other words, the customized humeral guidance G in the output may be used to form a complementary surface that is formed to mate with a specific anatomy of a specific patient, such as a concave surface that may be positioned on a corresponding convex surface, as discussed in more detail below.

Referring to FIG. 3, the electronic apparatus 110 includes a processor for receiving an input INPUT. The input INPUT includes patient information 34 and a preoperative shoulder image 32. The processor and software process the information, along with an implant that may be selected by the user concerning the nature of the shoulder surgery and other options regarding the patient's anatomy. The software generates data for controlling a 3D printing manufacturing process (not shown) of the customized guidance G. The data is configured to instruct 3D printing or other additive manufacturing processes. The 3D printing manufacturing process (not shown) includes a first operation of forming a mold as an additive manufacturing process and several subsequent operations of forming a customized guidance G in the mold. In particular, the electronic apparatus 110 uses the preoperative shoulder image 32 to configure a patient-specific surface of the customized guidance G as a complementary/negative surface, and design the guidance to be positioned according to the optimized fit determined by the surgeon or other user.

The electronic apparatus 110 also provides implant identification information I corresponding to the guidance G. For example, the electronic apparatus 110 provides the type of an implant suitable for preparing the guidance G to accommodate the humerus H.

In addition, the electronic apparatus 110 may provide patient output information P together with the customized guidance G and the implant identification information I.

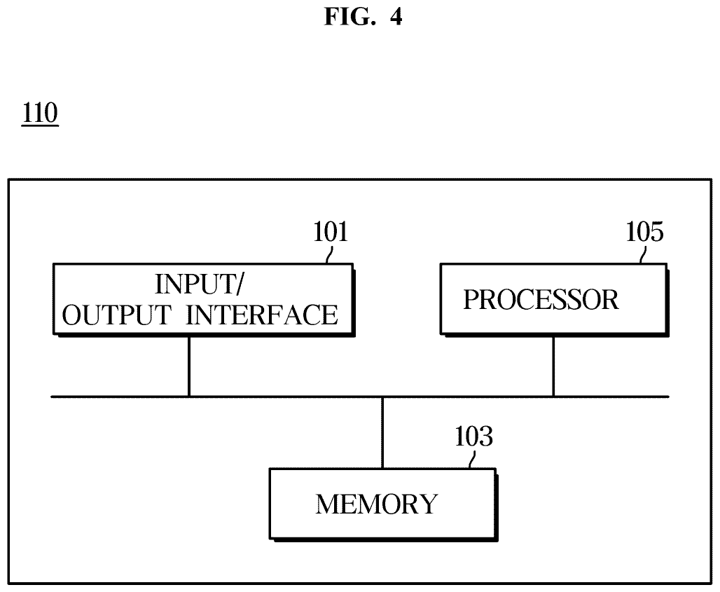

In FIG. 3, the input INPUT value of the electronic apparatus 110 and the output humeral guidance OUTPUT have been described. In FIG. 4, the configuration of the electronic apparatus 110 is described. FIG. 4 is a block diagram illustrating the configuration of the electronic apparatus according to an embodiment.

The electronic apparatus 100 shown in FIG. 4 includes only components related to the disclosed embodiment. Accordingly, those skilled in the art may understand that other general-purpose components may be included in addition to the components shown in FIG. 4.

As an example, the electronic apparatus 100 shown in FIG. 4 may be a server of a system for providing a collaborative service between a user and an operator, in 3D modeling for designing or manufacturing a patient-customized body model or a patient-customized medical device in relation to a medical service.

In this case, the system for providing a collaborative service may further include a plurality of operator devices or user devices similar to the electronic apparatus 110 shown in FIG. 4, as well as the server. Therefore, according to an embodiment, the system for providing a collaborative service may further include a network for supporting information transmission and reception between at least a part of the operator device or user device for a collaborative service between a user and an operator.

The electronic apparatus 110 according to the above embodiments may include a processor 105, a memory 103 for storing and executing program data, a permanent storage such as a disk drive, and a user input/output interface 101, such as a communication port for communicating with an external device, a touch panel, a key, a button, and the like. Methods implemented with software modules or algorithms may be stored on a computer readable recording medium as computer readable codes or program instructions executable on the processor. Here, the computer-readable recording media include a magnetic storage medium (e. g., a read-only memory (ROM), a random-access memory (RAM), a floppy disk, a hard disk, and the like), an optical readable medium (e. g., a compact disk (CD)-ROM, a digital versatile disk (DVD), etc.) and other recording media. The computer-readable recording medium may be distributed over computer systems connected through a network such that computer readable codes may be stored and executed in a distributed manner. The medium may be readable by a computer, stored in a memory, and executable in a processor.

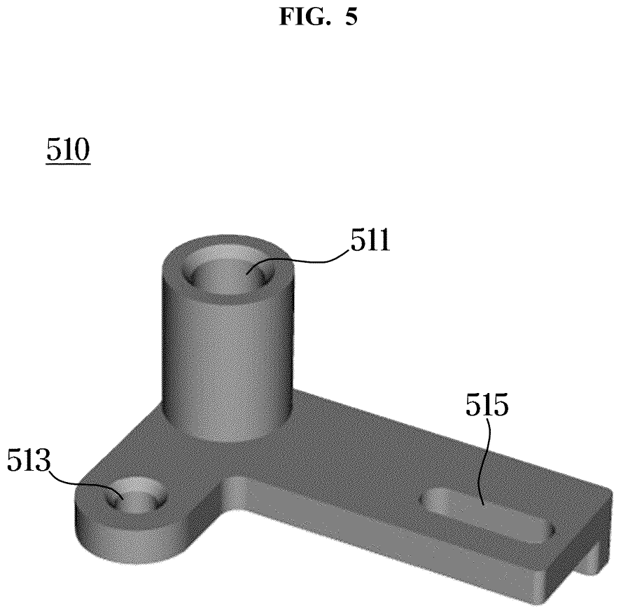

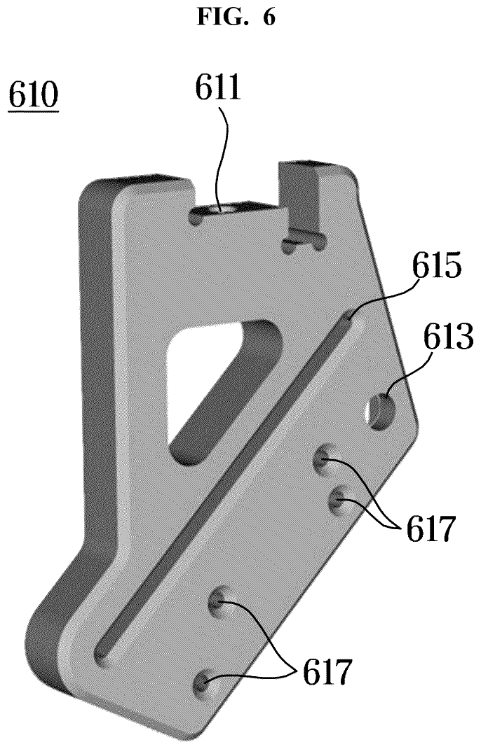

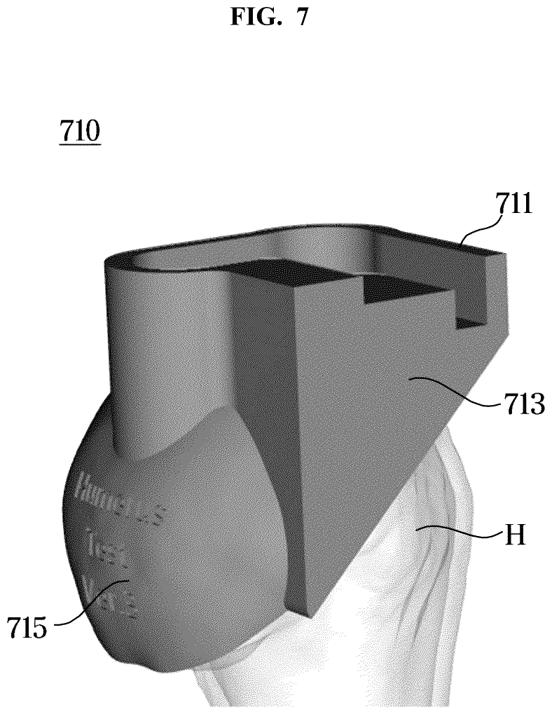

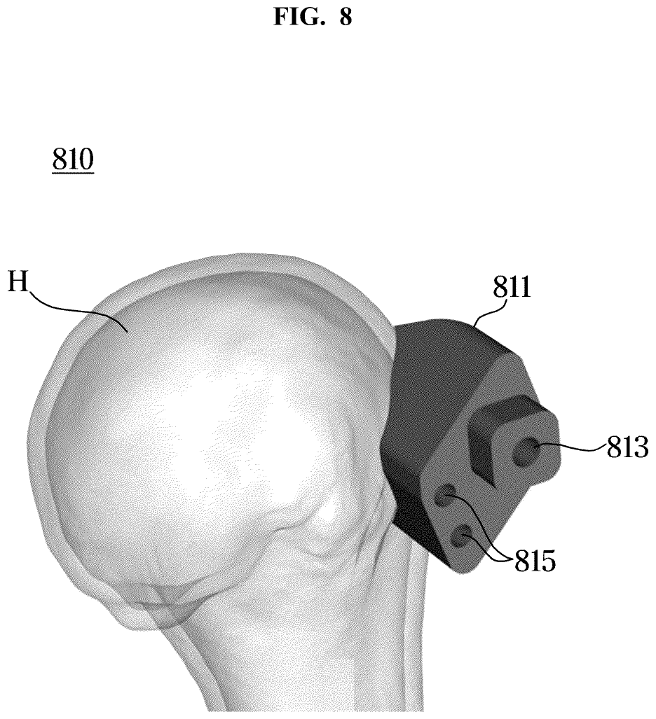

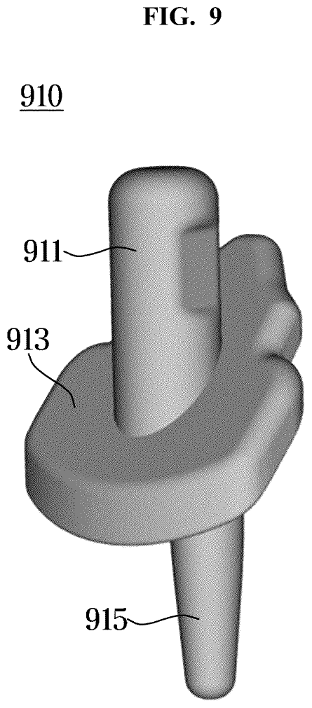

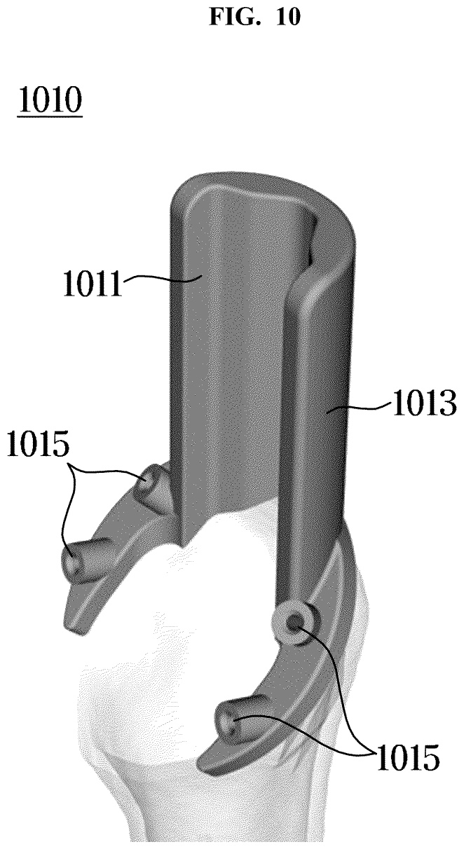

Hereinafter, with regard to the types and shapes of the humeral guidance G output by the electronic apparatus 110 of the present disclosure, that is, the types of the humeral guidance G that may be manufactured in a user-customized manner according to the present disclosure are described with reference to FIGS. 5 to 10. FIG. 5 is a perspective view of a reamer guidance according to an embodiment. FIG. 6 is a perspective view of a cutting guidance according to an embodiment. FIG. 7 is a side perspective view illustrating a first humeral guidance installed on the humerus according to an embodiment. FIG. 8 is a side perspective view illustrating a second humeral guidance installed on the humerus according to an embodiment. FIG. 9 is a perspective view illustrating a positioning pin guidance according to an embodiment. FIG. 10 is a perspective view illustrating a rail guidance installed on the humerus according to an embodiment.

A reamer guidance 510 illustrated in FIG. 5 includes a reamer insertion hole 511 into which a reamer is inserted to perform reaming, a screw hole 513 for fastening with a first humeral guidance shown in FIG. 7 described below, and a screw hole 515 for fastening with a cutting guidance shown in FIG. 6 described below.

In order that a reamer is inserted into the center of the head of the humerus to perform reaming, the reamer guidance 510 is placed in a stable position of the humerus head. However, the reamer guidance 510 may be supported to ensure insertion of a reamer after establishing a secure position relationship with the humerus by the pieces of guidance shown in FIGS. 5 to 8 described below being mutually being fastened to each other.

In addition, the screw hole 515 of the reamer guidance 510 has an elongated shape that may be adjusted according to the skeletal size of the humerus and fastened with the cutting guidance.

The reamer insertion hole 511 of the reamer guidance 510 has a certain depth in the z-axis direction such that the reamer may stably drill a hole in the humerus, and the screw hole 513 and the screw hole 515 are respectively provided in the x-axis direction and in the y-axis direction with respect to the reamer insertion hole 511, and the cutting guidance is erected in a direction perpendicular (x-axis) to the screw hole 515 on the lower surface of the reamer guidance 510 and is coupled to the screw hole 515. However, the shape of the reamer guidance 10 shown in FIG. 5 may vary.

Hereinafter, a cutting guidance 610 coupled to the reamer guidance 510 will be described. As shown in FIG. 6, the cutting guidance 610 includes a fastening groove 611 that is fastened to the reamer guidance 510, a screw hole 613 for fastening to a second humeral guidance shown in FIG. 8 to be described below, a saw hole 615 for performing sawing as planned, and a plurality of holes 617 for inserting pins into the humerus to fix the guidance.

The cutting guidance 610 is arranged on a side surface of the humerus and serves to assist in stably cutting a portion of the head of the humerus. Therefore, pins are inserted into the plurality of holes 617 for the cutting guidance 610 to be fixed to the humerus. However, pins are not inserted into all of the holes, but are inserted into at least one of the plurality of holes for fixation under the decision of the operating surgeon.

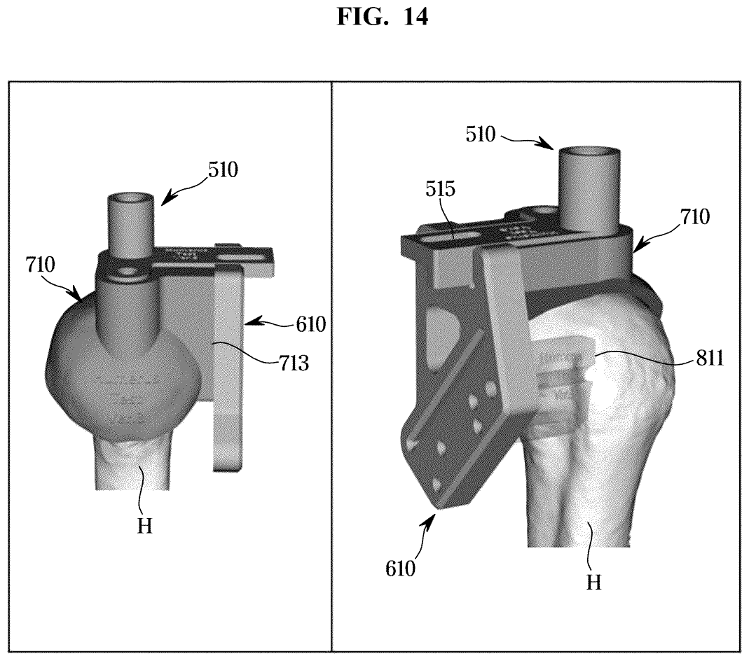

In addition, the cutting guidance 610 is fixed to the second humeral guidance through the screw hole 613, and the A method of arranging the second humeral guidance is described with reference to FIG. 14, to be presented below.

Next, FIG. 7 is a side perspective view illustrating a state in which the first humeral guidance 710 is installed on the humerus H according to an embodiment.

The first humeral guidance 710 has a fastening groove 711 on an upper side thereof that is press-fitted with the reamer guidance 510; a coupling surface 713 on one side thereof that is to be in contact with the cutting guidance 610, and a patient-fitting surface 715 that is tailored to accommodate a humerus head according to the individual humerus shape of the patient. The electronic apparatus 110 may receive a patient's shoulder image, perform 3D modeling and segmentation and 3D design, and design the customized guidance (S210 to S230) as described in FIG. 2, by which the patient-fitting surface is calculated (produced), and then manufacture the shape of the first humeral guidance 710 such that the first humeral guidance 710 is press-fitted to the reamer guidance 510 and contacts the cutting guidance 610 while including the calculated patient-fitting surface.

Next, FIG. 8 is a side perspective view illustrating a state in which a second humeral guidance 810 according to an embodiment is installed on the humerus H.

The second humeral guidance 810 includes a patient-fitting surface 811 that is a surface being in contact with the humerus head and fitted to the intertubercular groove of the humerus head, a screw fastening groove 813 that is a groove for fastening with the cutting guidance 610, and a plurality of pin holes 815 into which pins are inserted for fixing the second humeral guidance 810 to the humerus head.

Next, FIG. 9 is a perspective view illustrating a positioning pin guidance 910 according to an embodiment.

As shown in FIG. 9, the positioning pin guidance 910 refers to a pin for securing an implant insertion position before inserting an implant after sawing a portion of the humerus head using the cutting guidance 610 and removing the portion of the humerus head.

The positioning pin guidance 910 includes a handle 911 that allows a user to insert or remove the positioning pin guidance 910 into or from the humerus, a body 913 that allows the positioning pin guidance 910 to be seated on the humerus cut surface, and a pin 915 configured to be inserted into a reaming hole to secure a position.

Finally, FIG. 10 is a perspective view illustrating a state in which a rail guidance according to an embodiment is installed on the humerus. The rail guidance 1010 has a rail portion that serves as a guide such that a broach or implant is accurately inserted into the insertion position of the positioning pin guidance 910 in a desired direction and at a desired angle after the positioning pin guidance 910 secures the implant insertion position.

Specifically, the rail guidance 1010 includes an implant rail surface 1011, which is a rail portion formed to engage with the shape of a broach or an actual implant to be used, a body 1013 configured to support the implant rail surface 1011, and a plurality of holes 1015 into which pins for fixing the rail guidance 1010 are inserted.

The implant rail surface 1011 serves as a rail that allows the implant to be vertically inserted into the humerus while the back of the implant is rested against a groove generated by the positioning pin guidance 910.

In addition, although the plurality of holes 1015 of the rail guidance 1010 are illustrated as four holes in FIG. 10, the number of holes 1015 is not limited thereto, and the rail guidance 1010 may include more or few holes as long as the operating surgeon can insert pins appropriately to stably fix the rail guidance 1010 to the cut surface of the humerus.

In addition, in FIG. 10, only the case in which the plurality of holes 1015 are illustrated each as having a certain length and a certain angle such that the insertion direction and angle of the pin to be inserted are designated, but in certain cases, some of the plurality of holes may include holes without a depth such that the user may designate the insertion angle of the pin.

In FIGS. 5 to 10, the configurations of each guidance are described. The reamer guidance 510, the cutting guidance 610, the first humeral guidance 710, and the second humeral guidance 810 may be mutually coupled to form the first coupling guidance 1200 that helps the operating surgeon perform the reaming motion and the sawing motion conveniently and safely. Thereafter, subsequent to the reaming and sawing operations, the first coupling guidance is removed, and the positioning pin guidance 910 and the rail guidance 1010 are positioned on the cut surface of the humerus to form a second coupling guidance 1300.

All pieces of guidance described in FIGS. 5 to 10 may be manufactured by 3D printing through the operations shown in FIG. 2. In particular, the first humeral guidance and the second humeral guidance include patient-fitting surfaces and are manufactured as patient-customized guidance through the operations shown in FIG. 2.

However, the reamer guidance 510, the cutting guidance 610, and the positioning pin guidance 910 and the rail guidance 1010 may be pre-manufactured in various sizes, and in particular, the rail guidance 1010 may be pre-manufactured in various sizes in consideration of the broach or implant to be used.

Therefore, the operating surgeon may determine the reamer guidance, the cutting guidance, the positioning pin guidance, and the rail guidance to be used based on the patient's skeletal information, and the electronic apparatus 110 according to the present disclosure may manufacture a patient-customized guidance, which is the first humeral guidance and the second humeral guidance, based on the specification (size) information of the reamer guidance, the cutting guidance, the positioning pin guidance, and the rail guidance determined by the operating surgeon.

Therefore, the reamer guidance, the cutting guidance, the positioning pin guidance, and the rail guidance may be manufactured by 3D printing, or may be manufactured from metal. That is, when such a guidance is pre-manufactured in various sizes, the guidance may be provided in a form manufactured from metal without the need for 3D printing each time.

When the reamer guidance 510 is not manufactured through 3D printing but is pre-manufactured with metal, it may be pre-manufactured in various sizes such that the actual operating surgeon may select and use the size that fits the patient's affected area.

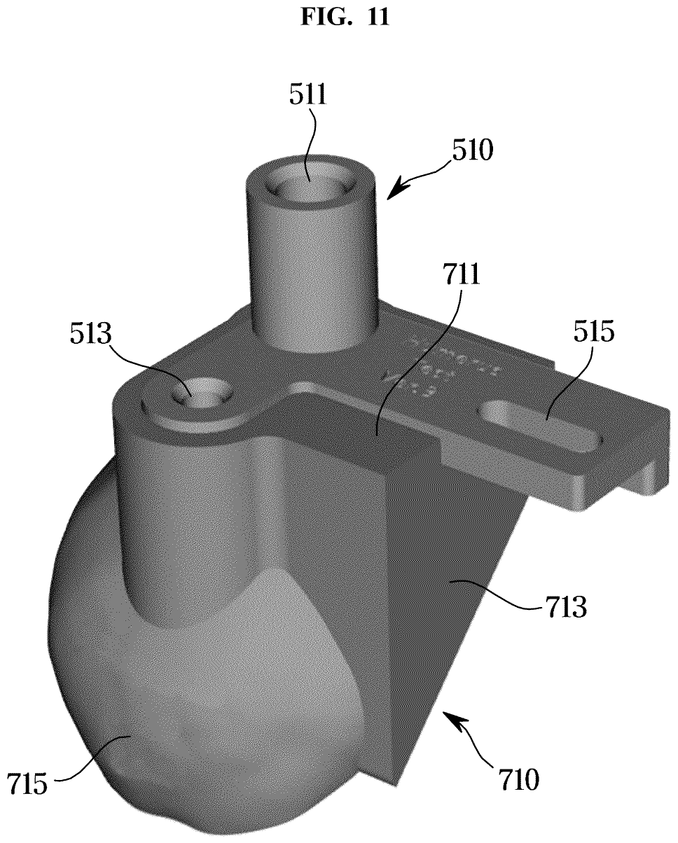

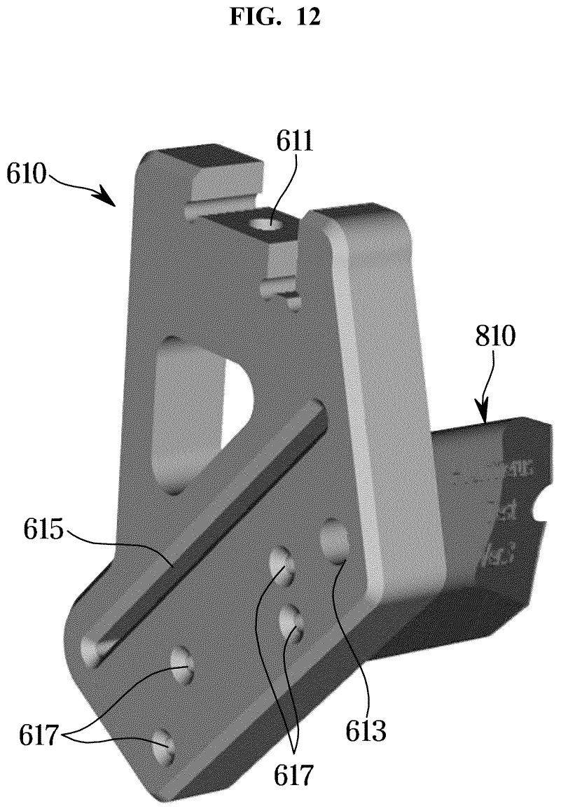

Hereinafter, a method of coupling a first coupling guidance 1200 is described with reference to FIGS. 11 to 16. FIG. 11 is a perspective view illustrating a first humeral guidance 710 coupled to a reamer guidance 510 according to an embodiment. FIG. 12 is a perspective view illustrating a second humeral guidance 810 coupled to a cutting guidance 610 according to an embodiment. FIG. 13 is a perspective view illustrating a state in which the first humeral guidance and the reamer guidance coupled as shown in FIG. 11 are installed on the humerus according to an embodiment. FIG. 14 is a perspective view illustrating a state in which the second humeral guidance and the cutting guidance coupled as shown in FIG. 12 are installed on and coupled to the humerus on which the first humeral guidance and the reamer guidance are installed as shown in FIG. 13.

First, referring to FIG. 11, the reamer guidance 510 and the first humeral guidance 710 are coupled to each other, and a screw is fastened to the screw hole 513 of the reamer guidance 510. Here, the reamer guidance 510 and the first humeral guidance 710 may be coupled in a press-fitted manner, and for the fixation thereof, a screw is fastened to the screw hole 513.

In addition, referring to FIG. 12, the second humeral guidance 810 and the cutting guidance 610 may be coupled to each other, and for the fixation thereof, a screw is fastened to the screw hole 613 of the cutting guidance 610.

Next, as shown in FIG. 13, the first humeral guidance 710 in the reamer guidance 510 and the first humeral guidance 710 coupled as shown in FIG. 11 is positioned to be mounted on the humerus head portion. That is, the first humeral guidance 710 is fastened such that the patient-fitting surface 715 designed as in FIG. 7 is completely in close contact with the coupling surface.

Next, referring to FIG. 14, the reamer guidance 510 and the cutting guidance 610 are coupled and then fastened. In this case, the reamer guidance 510 is in a state of being coupled to the first humeral guidance 710, and the cutting guidance 610 is in a state of being coupled to the second humeral guidance 810. The reamer guidance 510 and the cutting guidance 610 are fastened to be completely in close contact with the coupling surface 713 shown in FIG. 14, and are positioned such that the fitting surface of the second humeral guidance 810 that is pre-fastened to the cutting guidance 610 is completely fastened to the intertubercular groove of the humerus.

In addition, a screw may be fastened to the screw hole 515 to fit the size of the humerus such that the reamer guidance 510 and the cutting guidance 610 are completely fastened and fixed. That is, when the size of the humerus bone is small, the screw is fastened to pass through the fastening groove 611 at a position of the screw hole 515 close to the central axis of the reamer guidance 511, and when the size of the humerus bone is large, the screw is fastened to pass through the fastening groove 611 at a position of the screw hole 515 far from the central axis of the reamer guidance 511.

Next, while the first coupling guidance 1200 is seated to the humerus head, pins (for example, pins may be 1.2 mm pins) may be inserted into the plurality of holes 617 in the cutting guidance 610 for the fixation, thereby fixing the first coupling guidance 1200. Here, the cutting guidance 610 is illustrated as having four holes 617, but this is only one example, and the cutting guidance 610 may be manufactured to include more or few holes. In many cases, the cutting guidance 610 may be formed to include holes that allow the operating surgeon to insert only a desired number of pins at a desired position and a desired angle.

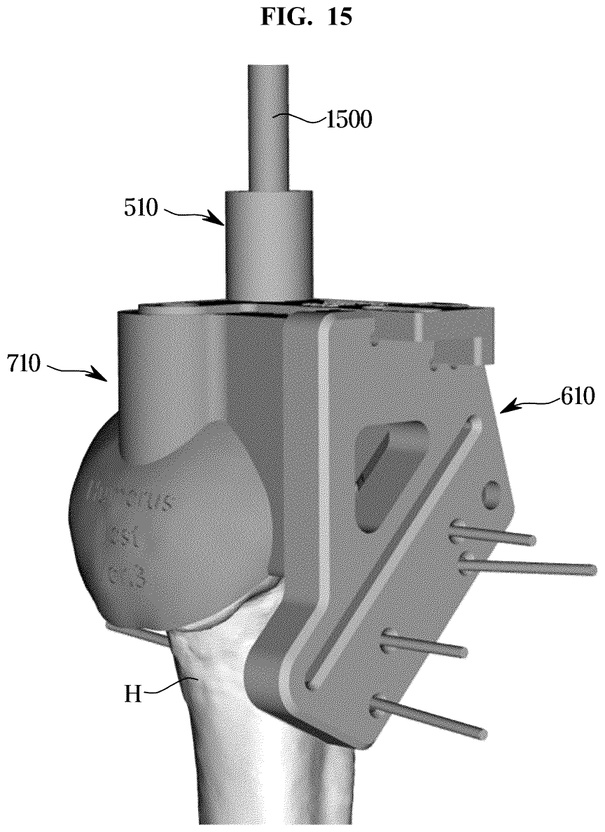

FIG. 15 is a perspective view illustrating an operation of inserting a reamer after the first humeral guidance, the reamer guidance, the second humeral guidance, and the cutting guidance are installed and coupled on the humerus as shown in FIG. 14.

Referring to FIG. 15, the operating surgeon may perform a reaming operation using a reamer 1500 on the humerus head to be reamed and cut while the first coupling guidance 1200 is fixed to the humerus head. By performing the reaming operation using the first coupling guidance 1200 customized for the patient, the reaming operation for inserting an implant into a desired position without shaking may be performed rapidly.

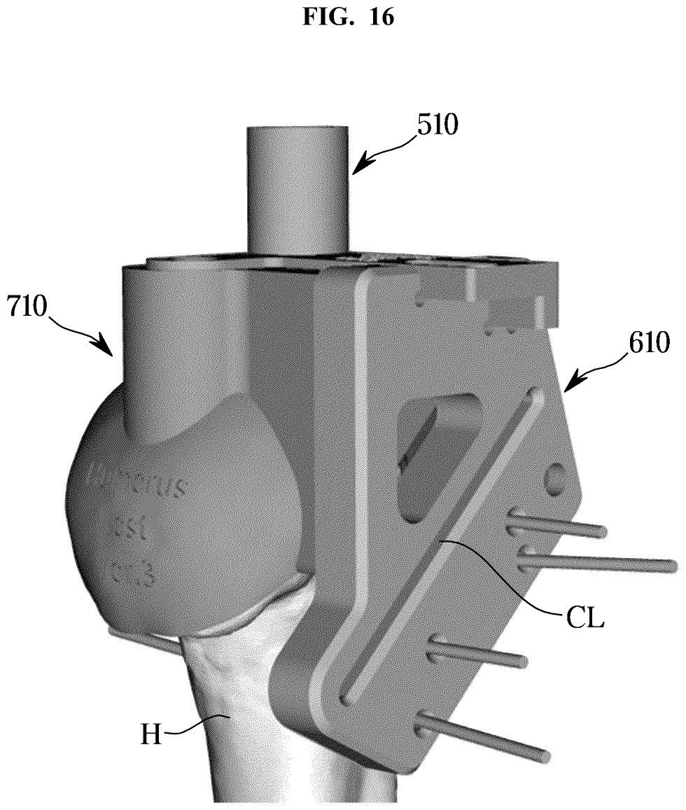

FIG. 16 is a perspective view illustrating a cutting operation of a saw after the first humeral guidance, the reamer guidance, the second humeral guidance, and the cutting guidance are installed and coupled on the humerus as shown in FIG. 14.

The operating surgeon may remove the reamer 1500, and then insert a saw into a saw hole 615 and perform the sawing operation along a cutting line CL as illustrated in FIG. 16. By performing the reaming operating and the sawing operations with a single combined guidance using the first combined guidance 1200 customized for the patient, the reaming position and the cutting position may be fixed, and the reaming and sawing operations for inserting the implant into the desired position may be performed in a shorter time compared to separately using reaming guidance and sawing guidance.

Hereinafter, a method of forming a second combined guidance 1300 that combines the positioning pin guidance 910 and the rail guidance 1010 after removing all pins after the reaming and sawing operations through the first combined guidance 1200 of FIGS. 13 to 16 will be described.

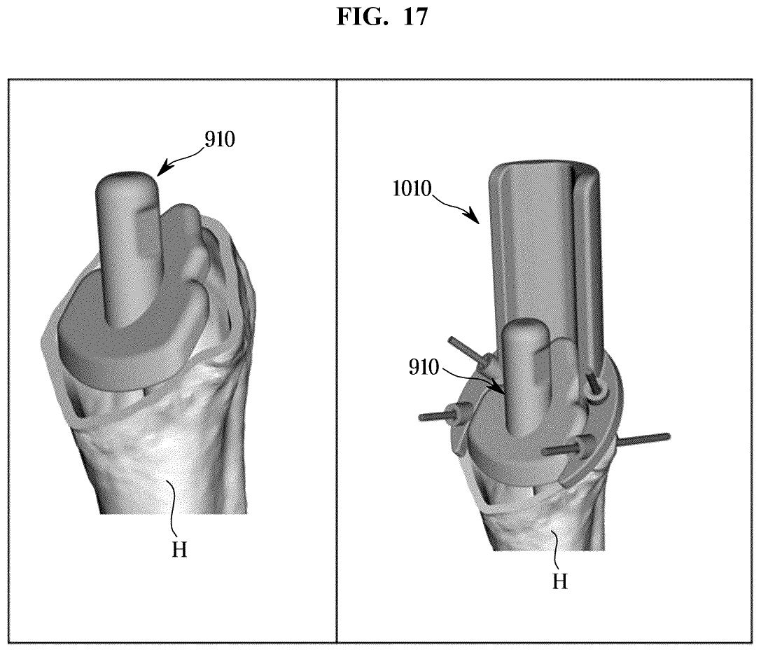

FIG. 17 is a perspective view illustrating an operation in which a positioning pin guidance according to an embodiment as shown in FIG. 9 is inserted into the humerus, and a rail guidance is seated on the positioning pin guidance.

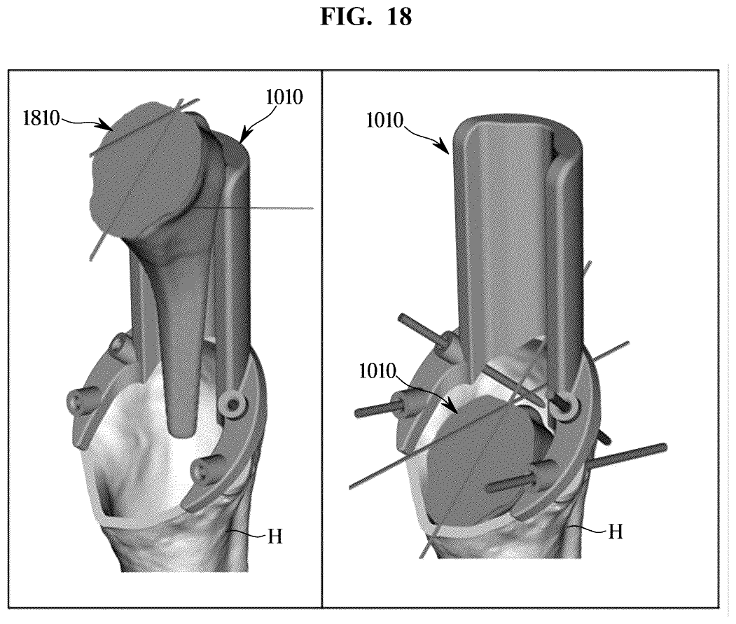

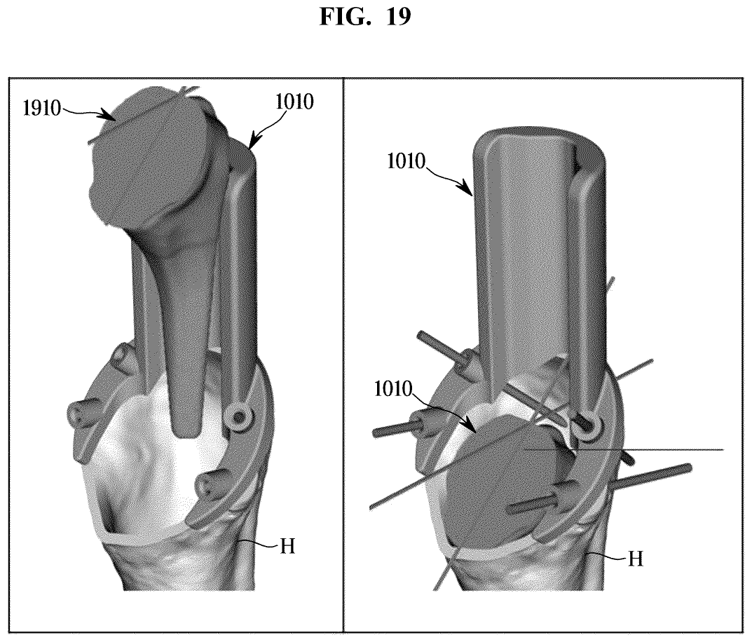

As illustrated in the left drawing of FIG. 17, the positioning pin guidance 910 is inserted into a reaming hole. Then, as illustrated in the right drawing of FIG. 17, the rail guidance 1010 is seated and a plurality of pins are inserted into the plurality of holes 1015 for the fixation. The rail guidance 1010 is intended to provide a guide for the broach or implant illustrated in FIG. 18 and FIG. 19 to be positioned at an accurate angle and direction with respect to the position of the positioning pin guidance 910.

FIG. 18 is a perspective view illustrating an operation in which a broach is inserted along the rail guidance according to an embodiment. That is, after removing the positioning pin guidance 910 shown in FIG. 17, the broach 1410 is inserted along the implant rail of the rail guidance 1010 to generate a groove and then is removed.

Next, FIG. 19 is a perspective view illustrating an operation in which a real implant is inserted along the rail guidance according to an embodiment.

In the same method as the above, the real implant may be inserted along the implant rail 1011 of the rail guidance 1010.

The examples of FIGS. 5 to 19 above are specific examples of one or more pieces of suggestion information according to the present disclosure, but the scope of the present disclosure is not limited thereto, and various other examples may exist.

The electronic apparatus 110 according to the above embodiments may include a processor, a memory for storing and executing program data, a permanent storage such as a disk drive, and a user interface apparatus, such as a communication port for communicating with an external apparatus, a touch panel, a key, a button, and the like. Methods implemented with software modules or algorithms may be stored on a computer readable recording medium as computer readable codes or program instructions executable on the processor. Here, the computer-readable recording media include a magnetic storage medium (e.g., a read-only memory (ROM), a random-access memory (RAM), a floppy disk, a hard disk, and the like), an optical readable medium (e.g., a compact disk (CD)-ROM, a digital versatile disk (DVD), etc.) and other recording media. The computer-readable recording medium may be distributed over computer systems connected through a network such that computer readable codes may be stored and executed in a distributed manner. The medium may be readable by a computer, stored in a memory, and executable in a processor.

The embodiments may be represented by functional block configurations and various processing operations. These functional blocks may be implemented with any number of hardware and/or software configurations that perform particular functions. For example, the embodiments may adopt integrated circuit configurations such as memory, processing, logic, look-up tables, etc., which may perform various functions by control of one or more microprocessors or by other control apparatuses. Similar to the way in which components may be implemented in software programming or software components, the present embodiments may be implemented in a variety of ways, including C, C++, Java, an assembler, python, and the like. Functional aspects may be implemented with algorithms running on one or more processors. In addition, the present embodiment may employ conventional techniques for electronic environment setting, signal processing, and/or data processing. Terms such as “mechanism”, “element”, “means”, “configuration” may be used broadly and are not limited to mechanical and physical configurations. The term may include the meaning of a series of routines of software in conjunction with a processor or the like.

The above-described embodiments are merely examples and other embodiments may be implemented within the scope of the claims described below.

1. A combined guidance for reaming and cutting, comprising:
a reamer guidance configured to guide reaming of a humerus;
a first humeral guidance press-fitted with the reamer guidance and having a surface tailored to a humerus head;
a second humeral guidance having a surface tailored to an intertubercular groove of the humerus head; and
a cutting guidance mutually coupled to the reamer guidance, the first humeral guidance, and the second humeral guidance.

2. The combined guidance of claim 1, wherein
the reamer guidance includes:
a first screw hole for fastening with the first humeral guidance;
a reamer insertion hole for reaming the humerus head; and
a second screw hole for fastening with the cutting guidance,
wherein the first humeral guidance includes a fastening groove for press-fitting with the reamer guidance.

3. The combined guidance of claim 2, wherein
the cutting guidance includes:
a plurality of pin holes for fixation to the humerus;
a sawing hole for sawing the humerus head as planned; and
a third screw hole for fastening with the second humeral guidance,
wherein the second humeral guidance includes:
a groove for fastening with the third screw hole of the cutting guidance; and
a plurality of pin holes for fixation to the humerus.

4. The combined guidance of claim 2, wherein the second screw hole of the reamer guidance for fastening with the cutting guidance has an elongated shape that allows fastening to the cutting guidance according to a skeletal size of the humerus.

5. The combined guidance of claim 1, wherein the reamer guidance and the cutting guidance include metal, and the first humeral guidance and the second humeral guidance include plastic.

6. A combined guidance for guiding positioning and implant installation, comprising:
a positioning pin guidance configured to guide a position of an implant on a cut surface of a humerus that is subjected to cutting; and
a rail guidance seated on the positioning pin guidance, and having a hole provided for fixing and coupling the rail guidance to a periphery of a head of the humerus that is subjected to cutting.

7. The combined guidance of claim 6, wherein the positioning pin guidance includes:
a handle allowing a user to insert or remove the positioning pin guidance into or from a reamed hole of the humerus;
a body allowing the positioning pin guidance to be seated on the cut surface of the humerus; and
a pin configured to be inserted into a pre-reamed hole to secure a position.

8. The combined guidance of claim 7, wherein the rail guidance includes:
an internal implant rail portion configured to engage with an implant or a broach desired to be installed;
an external body configured to support the internal implant rail portion; and
a plurality of holes provided for fixing and coupling the rail guidance to the periphery of the head of the humerus that is subjected to cutting.

9. The combined guidance of claim 8, wherein the positioning pin guidance is removable when the rail guidance is fixed and coupled to the periphery of the head of the humerus that is subjected to cutting.

10. The combined guidance of claim 9, wherein the plurality of holes of the rail guidance are pre-designed with pin insertion angles of the holes and hole depths of the holes.

11. A method of designing a guidance specific to a patient, the method comprising:
designing a reamer guidance configured to guide reaming of a humerus;
designing a first humeral guidance interference-fitted with the reamer guidance and having a surface tailored to a humerus head;
designing a second humeral guidance having a surface tailored to an intertubercular groove of the humerus head; and
designing a cutting guidance mutually coupled to the reamer guidance, the first humeral guidance, and the second humeral guidance.

12. The method of claim 11, comprising, before the designing of the reamer guidance, the first humeral guidance, the second humeral guidance, and the cutting guidance,
receiving image information about a body part of a target patient;
performing 3D modeling based on the received image information; and
providing a 3D modeling manufacturing function of a patient-customized medical device based on information about the 3D modeling.

13. The method of claim 12, wherein the designing of the reamer guidance configured to guide reaming of the humerus and the designing of the cutting guidance mutually coupled to the reamer guidance, the first humeral guidance, and the second humeral guidance include displaying, to an operator, predetermined specification information of the reamer guidance and predetermined specification information of the cutting guidance for selection.

14. The method of claim 13, further comprising:
when the operator inputs the specification information of the reamer guidance and the cutting guidance, and feedback information of the designed first humeral guidance and the designed second humeral guidance,
modifying the design of the guidance based on the specification information of the reamer guidance and the cutting guidance, and the feedback information of the first humeral guidance and the second humeral guidance that are input by the operator.

15. The method of claim 11, further comprising:
designing a positioning pin guidance configured to guide a position of an implant on a cut surface of a humerus that is subjected to cutting; and
designing a rail guidance configured to be seated on the positioning pin guidance, and having a hole provided for fixing and coupling the rail guidance to a periphery of a head of the humerus that is subjected to cutting.

16. The method of claim 15, comprising, before the designing of the positioning pin guidance and the rail guidance,
receiving image information about a body part of a target patient;
performing 3D modeling based on the received image information; and
providing a 3D modeling manufacturing function of a patient-customized medical device based on information about the 3D modeling.

17. The method of claim 16, wherein the designing of the positioning pin guidance configured to guide the position of the implant on the cut surface of the humerus, and the designing of the rail guidance configured to be seated on the positioning pin guidance and having the hole provided for fixing and coupling the rail guidance to the periphery of the head of the humerus that is subjected to cutting includes
displaying, to an operator, predetermined specification information of the positioning pin guidance and predetermined specification information of the rail guidance for selection.

18. The method of claim 16, wherein the designing of the rail guidance configured to be seated on the positioning pin guidance and having the hole provided for fixing and coupling the rail guidance to the periphery of the head of the humerus that is subjected to cutting includes:
inputting, by an operator, positions of a plurality of holes provided for fixing and coupling the rail guidance to the periphery of the head of the cut humerus, pin insertion angles of the holes, and hole depths of the holes;
modifying the design of the guidance based on information about the positions of the plurality of the holes. the pin insertion angles of the holes, and the hole depths of the holes that are input by the operator.
